(19) United States
(12) Patent Application Publication
(10) Pub. No.: US 2015/0202313 A1
(43) Pub. Date: Jul. 23, 2015

(54) Macromolecular Prodrugs for Hard Tissue and Methods of Use Thereof

(71) Applicants: New York Society for The Ruptured and Crippled Maintaining The Hospital for Special Surgery, New York, NY (US); Board of Regents of the University of Nebraska, Omaha, NE (US)

(72) Inventors: Dong Wang, Omaha, NE (US); Zhenshan Jia, Omaha, NE (US); Yijia Zhang, Omaha, NE (US); Steven R. Goldring, New York, NY (US); Aaron Daluiski, New York, NY (US)

(21) Appl. No.: 14/424,737

(22) PCT Filed: Sep. 3, 2013
(86) PCT No.: PCT/US13/57807

§ 371 Date: Feb. 27, 2015

Related U.S. Application Data
(60) Provisional application No. 61/695,543, filed on Aug. 31, 2012.

(51) Int. Cl. A61K 47/48; A61K 49/00; A61K 31/366

(52) U.S. Cl. CPC A61K 47/48215; A61K 47/481; A61K 31/366; A61K 47/488; A61K 49/00

(57) ABSTRACT

Methods and compositions for treating bone disorders are disclosed.

This application claims priority under 35 U.S.C. §119(e) to U.S. Provisional Patent Application No. 61/695,543, filed on Aug. 31, 2012. The foregoing application is incorporated by reference herein.

This invention was made with government support under Grant No. R01 AR053325 awarded by the National Institutes of Health. The government has certain rights in the invention.

### FIELD OF THE INVENTION

The present invention relates to the fields of bone disease and bone fracture. More specifically, the invention provides compositions and methods for treating bone diseases, disorders, and fractures.

### BACKGROUND OF THE INVENTION

Several publications and patent documents are cited throughout the specification in order to describe the state of the art to which this invention pertains. Each of these citations is incorporated herein by reference as though set forth in full.

With an aging population worldwide, osteoporosis and osteoporosis-related bone fractures have become a major public health challenge (Burge et al. (2007) J. Bone Miner. Res., 22:465-75). It was projected that by 2025, there will be over 3 million fractures in the US, with related expenditures of $25.3 billion per year (Dempster, D. W. (2011) Am. J. Manag. Care, 17 Suppl 6:S164-9; Rousculp et al. (2007) Value Health, 10:144-52). Aging and the incipient risk of osteoporosis result in the loss of bone mass and deterioration of bone quality predisposing to fracture. Importantly, the fracture repair process is delayed and less effective with aging, making the elderly population particularly susceptible to loss of productivity, as well as increased morbidity and mortality. Despite significant advances in the development of new drugs for osteoporosis, there is no systemic therapeutic agent approved by the US Food and Drug Administration (FDA) to enhance fracture healing.

Statins belong to a class of anti-lipidaemic drugs known as 3-hydroxy-3-methylglutaryl (HMG)-CoA reductase inhibitors. They have been widely used to treat cardiovascular diseases for decades (Laufs et al. (2000) Trends Cardiovasc. Med., 10:143-8.). In 1999, it was discovered that simvastatin (SIM) and lovastatin have a strong bone anabolic effect mediated through the BMP-2 pathway (Mundy, G. (1999) Science 286:1946-9; Garrett et al. (2002) Arthritis Res., 4:237-40). This finding was significant, as the discovery of a second indication for a marketed drug class would have warranted a fast-track regulatory approval process due to the favorable toxicology profile of the statins. Major efforts have been invested in attempting to validate this finding (Staal et al. (2003) J. Bone Miner Res., 18:88-96; Oxlund et al. (2001) Calcif. Tissue Int., 69:299-304; Yaturu, S. (2003) Endocr. Pract., 9:315-20; Jadhav et al. (2006) J. Pharm. Pharmacol., 58:3-18; Maeda et al. (2004) J. Cell Biochem., 92:458-71; Garrett et al. (2001) Curr. Pharm. Des., 7:715-36), but the results remain controversial (Park, J. B. (2009) Med. Oral Patol. Oral Cir. Bucal., 14:e485-8; Yao et al. (2006) J. Musculoskelet. Neuronal Interact., 6:277-83; von Stechow et al. (2003) BMC Musculoskelet. Disord., 4:8; Rejnmark et al. (2004) J. Bone Miner. Res., 19:737-44), which may be explained by the strong hepatotropic nature of the statins. Over 95% of orally administered SIM distributes to the liver (Jadhav et al. (2006) J. Pharm. Pharmacol., 58:3-18; Park, J. B. (2009) Med. Oral Patol. Oral Cir. Bucal., 14:e485-8), with less than 5% of the drug to distributing to the rest of the body. Increase of the oral administration doses can improve the distribution of SIM to the bone (Oxlund et al. (2001) Calcif. Tissue Int., 69:299-304; von Knoch et al. (2005) Biomaterials 26:5783-9; Ho et al. (2009) Eur. J. Clin. Invest., 39:296-303; Uzzan et al. (2007) Bone 40:1581-7), however, the high doses are impractical due to the associated systemic toxicities (Jadhav et al. (2006) J. Pharm. Pharmacol., 58:3-18; Park, J. B. (2009) Med. Oral Patol. Oral Cir. Bucal., 14:e485-8). To circumvent the hepatotropicity, transdermal application (Gutierrez et al. (2006) Osteoporos Int., 17:1033-42) and local delivery of statins to the fracture sites have been explored (Mundy, G. (1999) Science 286:1946-9; Ayukawa et al. (2009) Oral Surg. Oral Med. Oral Pathol. Oral Radiol. Endod., 107:336-42; Skoglund et al. (2007) BMC Musculoskelet. Disord., 8:98; Seto et al. (2008) J. Periodontal Res., 43:261-7; Lee et al. (2008) Biomaterials 29:1940-9; Tanigo et al. (2010) J. Control Release 143:201-6; Fukui et al. (2012) J. Bone Miner. Res., 27:1118-31; Pradeep et al. (2012) J. Periodontol., 83:1472-9; Morris et al. (2008) J. Periodontol., 79:1465-73; Ho et al. (2011) J. Orthop. Res., 29:1504-10; Garrett et al. (2007) J. Orthop. Res., 25:1351-7; Yoshinari et al. (2007) Dent. Mater. J., 26:451-6; Killeen et al. (2012) J. Periodontol., 83:1463-71; Lee et al. (2011) Mol. Pharmaceut., 8:1035-42; Chou et al. (2013) PLoS One 8:e54676). The doses and deposition site selection of the statins in these local delivery systems, however, are arbitrary with limited consideration of the pathophysiology of the fracture healing process. Accordingly, superior methods of delivering bone therapeutic agents such as statins are needed.

### SUMMARY OF THE INVENTION

In accordance with the present invention, macromolecular prodrugs for the delivery of therapeutic agents to a subject are provided. In a particular embodiment, amphiphilic compounds which spontaneously form micelles in aqueous solutions are provided. The amphiphilic compounds may comprise at least two, particularly at least three, hydrophobic therapeutic agents (e.g., bone anabolic agents) attached to the termini of a water-soluble polymer. The hydrophobic therapeutic agent may be attached to the water-soluble polymer via a linker, such as an alkyl. The linker may be biodegradable. For example, the linker may comprise an ester bond, which can be cleaved by an esterase in vivo. In a particular embodiment, the hydrophobic bone anabolic agent is a statin such as simvastatin or lovastatin. In another embodiment, the water-soluble polymer is polyethylene glycol or a derivative thereof, such as polyethylene glycol monomethylether.

In accordance with another aspect of the instant invention, micelles comprising the amphiphilic compounds are provided. As stated hereinabove, the amphiphilic compounds of the instant invention form micelles in aqueous solutions. The micelles of the instant invention may comprise one or more different amphiphilic compounds. For example, the amphiphilic compounds of the micelle may all be the same or may differ by having different therapeutic agents and/or different water-soluble polymers. In a particular embodiment, the micelle encapsulates an additional hydrophobic compound in its core. The additional hydrophobic compound is “free” in that it is not covalently linked to the amphiphilic compound. The core of the micelle may further comprise a hydrophobic therapeutic agent and/or a hydrophobic imaging agent. When the additional hydrophobic compound is a therapeutic agent such as a bone anabolic agent, the free therapeutic agent may be the same or different than the therapeutic agent of the amphiphilic compound. For example, the free therapeutic agent and the therapeutic agent of the amphiphilic compound may both be simvastatin. Alternatively, the therapeutic agent of the amphiphilic compound may be simvastatin and the free therapeutic agent may be lovastatin, an imaging agent, or any other hydrophobic compound. Compositions comprising a micelle of the instant invention and a pharmaceutically acceptable carrier are also provided. The compositions may comprise more than one different micelle (e.g., a micelle comprising simvastatin and a micelle comprising lovastatin or an imaging agent).

In accordance with another aspect of the instant invention, methods of increasing bone mass in a subject are provided. The method comprises administering at least one micelle of the instant invention to the subject. The instant invention also encompasses methods of treating a bone fracture in a subject comprising administering at least one micelle of the instant invention to the subject. Methods of treating an autoimmune disease in a subject are also provided. The methods comprise administering at least one micelle of the instant invention to the subject. The micelles of the instant invention may be administered directly to the desired site or intravenously in the above methods.

### BRIEF DESCRIPTION OF THE DRAWINGS

FIG. 1 provides a schematic of the synthesis of the shamrock-like amphiphilic macromolecular prodrug SIM-mPEG. Reaction a: PPh₃ (5 eq), I₂ (4 eq), DCM, RT, 24 hours, 79.1%; Reaction b: NaN₃ (20 eq), DMF, 100° C., 24 hours, 93.4%; Reaction c: 3-butyn-1-ol (6 eq), TsOH.H₂O (0.1 eq), RT, 3 hours, 30.3%; Reaction d: succinic anhydride (6 eq), Et₃N (4 eq), DMAP (0.4 eq), 45° C., 20 hours; Reaction e: DCC (2.5 eq), SIM (2.5 eq), DMAP (0.06 eq), 0° C., 1.5 hours, 55.1% for two steps; Reaction f: 6 (2.5 eq), 3 (1 eq), CuSO₄.5H₂O (1 eq), L-ascorbic acid sodium salt (2 eq), t-BuOH/H₂O, 60 hours, 77.3%.

FIGS. 2A and 2B show targeting of SIM/SIM-mPEG to the fracture site. FIG. 2A provides near infrared optical imaging demonstrated the targeting of IRDye® 800CW-labeled SIM/SIM-mPEG to the fracture site in the right femur of the osteotomized Swiss-Webster mice (Supine position). FIG. 2B provides a graph of the semi-quantitative analysis of NIR signal intensity (from IRDye® 800CW-labeled SIM/SIM-mPEG micelles) at the selected region of interest. Data presented as mean±SD (n=3). The signal intensity differences between the fractured leg and the contralateral intact leg were statistically significant (t-test, P<0.05).

FIG. 3 provides representative micro-CT images of the fractured femurs. At 21 days post-fracture the SIM/SIM-mPEG treated group exhibited more extensive mineralization of the fracture callus than Con and SIMA treated groups. The white bars inside the bone marrow in the coronal and sagittal panels and the white solid circles in the transaxial panels were the steel rods utilized for bone stabilization prior to the osteotomy. The outside diameter of the rod is 0.5 mm.

FIG. 4 provides graphs of the quantitative morphometric indices of callus tissue obtained from micro-CT analysis. The SIM/SIM-mPEG group demonstrates increased callus volume, thickness and better bone tissue organization than SIM and Con. Data is expressed as mean±SD. *P<0.05 vs. Con, **P<0.01 vs. Con.

FIG. 5 provides images of Safranin O stained histological sections of callus from the different treatments groups. The three panels on the right are an enlarged presentation (×10) of the outlined region in the left panels (×2). The callus sections of the SIM/SIM-mPEG group were devoid of chondrocytes and cartilage matrix and exhibited extensive woven and lamellar bone formation. Bar=1 mm (left panels) or 0.2 mm (right panels).

### DETAILED DESCRIPTION OF THE INVENTION

The recently reported Extravasation through Leaky Vasculature and Inflammatory cell-mediated Sequestration (ELVIS) mechanism (Wang et al. (2011) Mol. Pharmaceut., 8:991-3; Ren et al. (2011) Mol. Pharmaceut., 8:1043-51; Yuan et al. (2012) Arthritis Rheumat., 64:4029-39; Yuan et al. (2012) Adv. Drug Del. Rev., 64:1205-19) provides a unique opportunity to systemically target therapeutic agents to inflammatory pathologies, including fractures. As hematoma and acute inflammation are key initial pathological features of bone fractures, the leaky vasculature associated with the fracture facilitates the extravasation of systemically administered colloids at the fracture site. Before being cleared via lymphatic drainage, the colloids are quickly internalized and sequestered by the local resident cells at the fracture site, which provides a novel retention mechanism for the drug delivery system. The retained drug within the formulation can then be gradually released from cells at the fracture sites to modulate the healing process.

Simvastatin (SIM), a widely used anti-lipidaemic drug, has been identified as a bone anabolic agent. Its poor water solubility and the lack of distribution to the skeleton, however, have limited its application in the treatment of bone metabolic diseases. Herein, a shamrock-like amphiphilic macromolecular prodrug of simvastatin was designed and synthesized to overcome these challenges. More specifically, the design and synthesis of a shamrock-like, amphiphilic prodrug of simvastatin (SIM-mPEG) based on ω-methoxypolyethylene glycol (mPEG) is provided. The polyethylene glycol (PEG)-based prodrug can spontaneously self-assemble to form micelles. The use of SIM trimer as the prodrug's hydrophobic segment allows easy encapsulation of additional free SIM (SIM/SIM-mPEG). Using in vivo optical imaging, the systemically administered micelle prodrug formulation was found to passively target the bone fracture sites associated with hematoma/inflammation. The treatment study further validates the micelle formulation's therapeutic efficacy in promoting bone fracture healing as demonstrated by micro-CT and histological analyses. Because of the excellent clinical safety profile of SIM, this macromolecular prodrug-based micelle formulation can be used in clinical management of impaired fracture healing.

The amphiphilic compounds (micelle forming units) of the instant invention have the general formula of (therapeutic agent)ₙ-water-soluble polymer, where “n” represents the number of therapeutic agents attached to a single water-soluble polymer. The therapeutic agent of the compound is hydrophobic, thereby rendering the compound amphiphilic (the water-soluble polymer is hydrophilic). In a particular embodiment, n is greater than one, particularly 3 or more. In a particular embodiment, n=3. Typically, the therapeutic agent(s) are attached to the water-soluble polymer by a linker. The therapeutic agents attached to the water-soluble polymer may all be the same or may be different. The therapeutic agents are also generally attached to one termini of the water-soluble polymer.

While polyethylene glycol (PEG) and derivatives thereof (e.g., monomethylether PEG (mPEG)) are exemplified throughout the instant application, other water-soluble polymers may be used in the compounds of the instant invention. Water-soluble polymers of the instant invention include, but are not limited to, polymers comprising a methyl acrylamide backbone, N-(2-hydroxypropyl)methacrylamide (HPMA) copolymers and derivatives, polyethylene glycol polymers including branched or block copolymers, poly oxazolines (e.g., poly (2-oxazoline)), polyglutamic acid, polyaspartic acid, dextran, chitosan, cellulose and its derivatives, starch, gelatin, hyaluronic acid and its derivatives, and copolymers and derivatives thereof. Water-soluble polymers of the instant invention include, but are not limited to, polymers or copolymers (e.g., block or graft) of the following monomers: N-isopropylacrylamide (e.g., PNIPAm), acrylamide, N,N-dimethylacrylamide, N-vinylpyrrolidone (e.g., PVP), vinyl acetate (e.g., resulting polymer hydrolyzed into polyvinyl alcohol or PVA), hydroxyethylmethacrylate (e.g., PHEMA), 2-oxazoline, 2-methacryloxyethyl glucoside, acrylic acid, methacrylic acid, vinyl phosphonic acid, styrene sulfonic acid, maleic acid, 2-methacrylloxyethyltrimethylammonium chloride, methacrylamidopropyltrimethyl-ammonium chloride, methacryloylcholine methyl sulfate, N-methylolacrylamide, 2-hydroxy-3-methacryloxypropyltrimethyl ammonium chloride, 2-methacryloxyethyl-trimethylammonium bromide, 2-vinyl-1-methylpyridinium bromide, 4-vinyl-1-methyl-pyridinium bromide, ethyleneimine, (N-acetyl)ethyleneimine, (N-hydroxyethyl)ethyleneimine and/or allylamine. In a particular embodiment, the polymer comprises about 1 to about 1000 monomer units, particularly about 10 to about 500.

In a particular embodiment, the water-soluble polymer is a polymer or copolymer comprising polyethylene glycol or a derivative thereof (e.g., heterofunctionalized PEGs such as polyethylene glycol monomethylether, HO-PEG-NH₂, HO-PEG-SH, etc.). In a particular embodiment, the polyethylene glycol derivative comprises at least one chemical moiety (e.g., —SH, —NH₂, —OH, —COOH, etc.) at a termini (or both termini) to allow for chemical modification. In a particular embodiment, the water-soluble polymer is polyethylene glycol or polyethylene glycol monomethylether. Typically, the polyethylene glycol polymer will have a molecular weight of less than about 7 kDa, less than about 5 kDa, less than about 3 kDa, less than about 2.5 kDa, or less than about 2 kDa. Typically, the polyethylene glycol polymer will also have a molecular weight of greater than about 0.5 kDa, or greater than about 1 kDa. In a particular embodiment, the polyethylene glycol polymer has a molecular weight of about 1 kDa to about 5 kDa, about 1 kDa to about 3 kDa, or about 1 kDa to about 2 kDa.

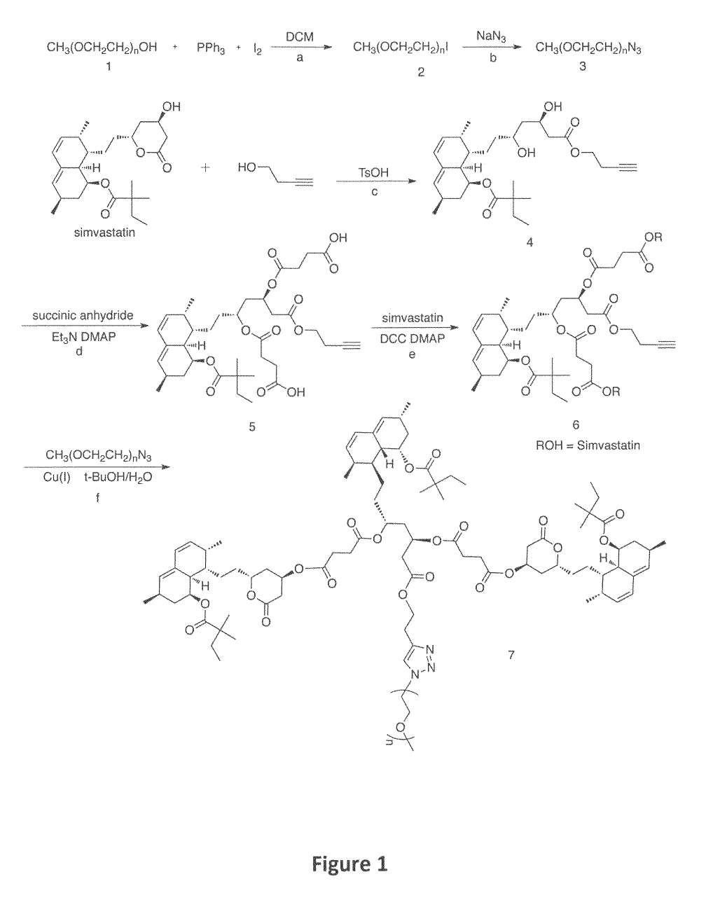

As stated hereinabove, the therapeutic agents will typically be attached to the water-soluble polymer by a linker. Each therapeutic agent may be linked directly to the water-soluble polymer via a linker. In a particular embodiment, a single linker (e.g., a branched linker) is attached to the water-soluble polymer and the therapeutic agents are attached (e.g., directly and/or via a linker) to the linker attached to the water-soluble polymer. For example, compound 7 of FIG. 1 comprises a single linker attached to the water-soluble polymer mPEG wherein the three simvastatin molecules are attached to the single linker attached to the water-soluble polymer. The therapeutic agents may be attached to the linker at different positions or branch from the linker from a single position (e.g., star formation).

Linkers are known in the art and the skill artisan may select a spacer based on length, reactivity, flexibility and the like. Linkers are chemical moieties comprising a covalent bond or a chain of atoms that covalently attaches at least two compounds. The linker can be linked to any synthetically feasible position of the compounds, but preferably in such a manner as to avoid blocking the compounds desired activity.

Exemplary linkers may comprise at least one optionally substituted; saturated or unsaturated; linear, branched or cyclic alkyl group or an optionally substituted aryl group. In a particular embodiment, the linker may contain from 0 (i.e., a bond) to about 500 atoms, about 1 to about 100 atoms, or about 1 to about 50 atoms. In a particular embodiment, the linker is an alkyl, particularly an alkyl having 1 to about 10 carbons in the main chain. The linker may also be a polypeptide (e.g., from about 1 to about 20 amino acids, particularly about 1 to about 10 amino acids). The linker may be non-degradable and can be a covalent bond or any other chemical structure which cannot be cleaved under physiological environments or conditions. The linker may be biodegradable under physiological environments or conditions (e.g., the linker may be cleaved upon a stimulus including, but not limited to, presence of light of certain wavelengths, changes in pH, presence of a specific enzyme (protease) activity (e.g., esterases, cathespins (e.g., cathepsin K), MMPs, etc.), changes in oxygen levels, etc.). For example, the linker may contain a bond which is cleavable under acidic pH (e.g., pH<6, particularly <5.5). Examples of pH sensitive linkers comprise, without limitation, a hydrazone bond, acetal bond, cis-aconityl spacer, phosphamide bond, silyl ether bond, etc.

The water-soluble polymers of the instant invention may also be attached to one or more targeting moieties. The targeting moieties may be attached to the opposite termini of the therapeutic agent. Preferably, the targeting moiety is a compound which preferentially accumulates in/on hard tissue (e.g., bone) rather than any other organ or tissue in vivo. Targeting moieties of the instant invention include, without limitation, folic acid, mannose, bisphosphonates (e.g., alendronate), pyrophosphate and derivatives thereof, quaternary ammonium groups, tetracycline and its analogs, sialic acid, malonic acid, N,N-dicarboxymethylamine, 4-aminosalicyclic acid, 5-aminosalicyclic acid, antibodies or fragments or derivatives thereof specific for hard tissue (e.g., Fab, humanized antibodies, and/or single chain variable fragment (scFv)), and peptides (e.g., peptides comprising about 2 to about 100 (particularly 6) D-glutamic acid residues, L-glutamic acid residues, D-aspartic acid residues, L-aspartic acid residues, D-phosphoserine residues, L-phosphoserine residues, D-phosphothreonine residues, L-phosphothreonine residues, D-phosphotyrosine residues, and/or L-phosphotyrosine residues). The targeting moiety may be linked to the water-soluble polymer directly or by a linker. In a particular embodiment, less than 100% of the water-soluble polymers of the micelle comprise a targeting moiety. For example, less than about 50%, less than about 40%, less than about 30%, less than about 20%, or less than about 10% of the water-soluble polymers of the micelle have a targeting moiety.

The therapeutic agent of the compounds of the instant invention may be any agent that imparts a benefit to subject having a bone-related disease, disorder, and/or fracture. One or more different therapeutic agents may be attached to a single water-soluble polymer. Examples of therapeutic agents include, without limitation, agents which increase bone growth and/or repair bone, agents which inhibit or treat a microbial infection (e.g., an antimicrobial or antibiotic), agents which alleviate pain (e.g., pain management drug such as NSAIDs, opiods, etc.), agents which regulate bone metabolism, and agents (e.g., chemotherapeutic agents) that modulate the activity and/or growth of primary or metastatic bone tumors. In a particular embodiment, the therapeutic agent is a bone anabolic agent. A bone anabolic agent is an agent which induces or increases bone forming activity. Bone anabolic agents include without limitation: HMG-CoA reductase inhibitors such as statins (e.g., simvastatin, mevastatin, lovastatin, pravastatin, velostatin, fluvastatin, cerivastatin, mevastatin, dalvastatin, fluindostatin, atorvastatin, etc., particularly lovastatin or simvastatin), prostaglandins (e.g., prostaglandin E1 or E2 and analogs thereof), prostaglandin agonists (e.g., prostaglandin E receptor agonist), strontium (e.g., strontium salts such as strontium ranelate), rho-kinase inhibitors (e.g. hydroxyfasudil), glycogen synthase kinase-3 β (GSK3β) inhibitors (e.g., lithium, 603281-31-8 (Kulkarni et al. (2006) J. Bone Miner. Res., 21:910-20)), secreted frizzled-related protein 1 (Sfrp1) inhibitors, herbal medicine (e.g., salvianolic acid A salvianolic acid B, icariin, osthole, etc.), cathepsin K inhibitor (e.g., opdanacatib), resolvins (e.g., WO/2007/061783), bone morphogenic protein (BMP)/transforming growth factor-β family members (e.g., BMP-2, BMP-4, BMP-6, BMP-7, etc.), inhibitors of the BMP pathway inhibitors (e.g. noggin, cytokines (e.g. IL-18, IL-22), oncostatin M, etc.), wnt inhibitors, and parathyroid hormone and fragments thereof. In a particular embodiment, the therapeutic agent is a statin, particularly simvastatin.

In a particular embodiment, the amphiphilic compound of the instant invention is compound 7 of FIG. 1 or a derivative/modification thereof. For example, compound 7 may be modified in one or more of the following ways. One, two, or all three of the simvastatin of the compound 7 may be replaced with another therapeutic agent such as another bone anabolic agent, particularly another statin. The water soluble polymer of compound 7 may also be replaced with another water-soluble polymer as described hereinabove. Additionally, the linkers of compound 7 may be replaced with other linkers as described hereinabove (e.g., with longer or shorter alkyls).

As explained throughout the application, the compounds of the instant invention form micelles in aqueous solutions. The micelles of the instant invention are preferably not cross-linked. The micelles of the instant invention may comprise one or more different compounds. For example, the micelles may comprise a first compound having a first water-soluble polymer attached to a first therapeutic agent and a second compound having a second water-soluble polymer attached to a second therapeutic agent. In a particular embodiment, the first and second therapeutic agents are different (e.g., two different statins or a statin and a pain reliever, etc.). In a particular embodiment, the first and second water-soluble polymers are the same (though they could be different).

In a particular embodiment, the micelles of the instant invention further comprise an additional therapeutic agent in the hydrophobic core of the micelle. The additional therapeutic agent is not attached or linked to the water-soluble polymer (i.e., the additional therapeutic agent is “free”). The additional therapeutic agent may be the same or different from the therapeutic agent attached to the water-soluble polymer. For example, and as shown in the Example hereinbelow, the micelle may comprise simvastatin linked to a water-soluble polymer and further comprise free simvastatin in the core of the micelle. When the additional therapeutic agent is different than the therapeutic agent attached to the water-soluble polymer, the therapeutic agents may be the same class of compounds (e.g., both may be bone anabolic agents or both may be statins) or they may be from different classes (e.g., a bone anabolic agent and a pain reliever). In a particular embodiment, at least one of the therapeutic agents in the micelle is a bone anabolic agent.

While generally the compounds and micelles of the instant invention will comprise a therapeutic agent as described hereinabove, the agent attached to the water-soluble polymer or encapsulated in the micelle can be any hydrophobic compound. For example, a therapeutic agent of the instant invention can be replaced with an imaging agent (e.g., the agent attached to the water-soluble polymer and/or the agent encapsulated in the micelle can be an imaging agent). Examples of imaging agents include, for example, compounds useful for optical imaging, magnetic resonance imaging (MRI), positron emission tomography (PET), computerized tomography (CT), gamma-scintigraphy imaging, and the like. Such agents are well-known to those of skill in the art. Imaging agents include, without limitation, radioisotope, isotopes, biotin and derivatives thereof, gold (e.g., nanoparticles), optical imaging agents (e.g., near IR dyes (e.g., IRDye® 800CW) phorphyrins, anthraquinones, anthrapyrazoles, perylenequinones, xanthenes, cyanines, acridines, phenoxazines, phenothiazines and derivatives thereof), chromophore, fluorescent compounds (e.g., Alexa Fluor® 488, fluorescein, rhodamine, DiI, DiO, and derivatives thereif), MRI enhancing agents (for example, DOTA-Gd3⁺ (1,4,7,10-tetraazacyclododecane-1,4,7,10-tetra (acetic acid)), DTPA-Gd3⁺ (gadolinium complex with diethylenetriamine pentaacetic acid), etc.), paramagnetic or superparamagnetic ions (e.g., Gd(III), Eu(III), Dy(III), Pr(III), Pa(IV), Mn(II), Cr(III), Co(III), Fe(III), Cu(II), Ni(II), Ti(III), and V(IV)), PET compounds labeled or complexed with ¹¹C, ¹³N, ¹⁵O, ¹⁸F, ⁶⁴Cu, ⁶⁸Ga or ⁸²Rb (e.g., ¹⁸F-FDG (fluorodeoxyglucose)), CT compounds (for example, iodine or barium containing compounds, e.g., 2,3,5-triiodobenzoic acid), and gamma or positron emitters (for example, ^{99m}Tc, ¹¹¹In, ¹¹³In, ¹⁵³Sm, ¹²³I, ¹³¹I, ¹⁸F, ⁶⁴Cu, ²⁰¹Tl, etc.).

The instant invention also encompasses compositions comprising at least one compound of the instant invention and at least one carrier. The instant invention also encompasses compositions comprising at least one micelle comprising at least one compound of the instant invention and at least one carrier (e.g., a pharmaceutically acceptable carrier). The compositions of the instant invention may further comprise at least one other therapeutic agent and/or imaging agent.

The micelles or compositions of the instant invention may be administered, in a therapeutically effective amount, to a subject in need thereof for the treatment and/or imaging of a bone disease or disorder. For example, the micelles or compositions of the instant invention may be administered to a subject for the treatment of impaired fracture healing (e.g., osteoporosis related bone fracture), for bone regeneration, and for fusion associated with orthopedic surgical procedures such as spinal fusion and other bone defects. In a particular embodiment, the micelles or compositions of the instant invention are administered, in a therapeutically effective amount, to a subject in need thereof for the treatment and/or imaging of a bone fracture. In a particular embodiment, at least one other therapeutic agent and/or imaging agent is administered separately from the micelles or compositions of the instant invention (e.g., sequentially or concurrently). In a particular embodiment, more than one micelle or composition of the instant invention is administered to the subject. For example, a micelle comprising a first therapeutic agent may be administered with (e.g., sequentially or concurrently) a micelle comprising a second therapeutic agent and/or an imaging agent.

The micelles or compositions of the instant invention may also be used to treat or inhibit an autoimmune and/or inflammatory disease. In a particular embodiment, the micelle comprises a statin, particularly simvastatin, as a therapeutic agent. Rho GTPases and their downstream effectors Rho-associated kinases (ROCKS) converge on multiple pathophysiological pathways involved in inflammatory and autoimmune diseases. Studies in the vascular system, kidney, and immune system have identified physiological and pathophysiological roles for Rho-Rock in specific cellular processes including proliferation, contraction, growth, matrix synthesis, apoptosis and migration. In addition, Rho-ROCK pathway agonists have been implicated in endothelial permeability and angiogenesis and their relevance documented in a number of studies indicating the protective effect of ROCK inhibitors in a variety of in vivo and in vitro models of cardiovascular and progressive renal disease (reviewed in Komers, E. (2010) Curr. Opin. Nephrol. Hypertens., 20:77-83). There is also evidence that inhibition of ROCK is the underlying mechanism by which statins or HMG-CoA reductase inhibitors exert their therapeutic effect beyond cholesterol in slowing the progression of atherosclerotic cardiovascular disease (Zhou et al. (2011) Trends Pharmacol. Sci., 32:167-73). More recently, ROCKs have been implicated in the regulation of the differentiation of murine Th17 cells and the production of interleukin-17 (IL-17) and IL-21, two cytokines associated with systemic lupus erythematosus (Isgro et al. (2013) Arthritis Rheum., 65:1592-602). A relationship between statins and regulation of the ROCK pathway has been more directly addressed. Simvastatin was shown to abrogate neutrophil adhesive properties in association with the inhibition of Mac-1 integrin expression and modulation of Rho kinase activity (Silveira et al. (2013) Inflamm. Res., 62:127-32). It was also shown that in the presence of a TNF-α inflammatory stimulus, neutrophils displayed a rapid and significant enhancement in their adhesive properties that was abrogated by preincubation of the cells with simvastatin. Accordingly, simvastatin will inhibit the differentiation of Th17 cells via inhibition of the Rho-ROCK pathway. The magnitude and duration of the inhibitory effects will be enhanced with the micelles of the instant invention due to their unique cellular uptake, sustained retention, and regulated drug release kinetics. Further, the favorable PK/PD characteristics of the micelles of the instant invention will result in attenuation of nephritis and associated organ system pathology in a murine model of systemic lupus erythematosus.

As used herein, the term “autoimmune disease” refers to the presence of an autoimmune response (an immune response directed against an auto- or self-antigen) in a subject. Autoimmune diseases include diseases caused by a breakdown of self-tolerance such that the adaptive immune system responds to self antigens and mediates cell and tissue damage. In a particular embodiment, autoimmune diseases are characterized as being a result of, at least in part, a humoral and/or cell-mediated immune response. Examples of autoimmune disease include, without limitation, rheumatoid arthritis, type 1 diabetes, systemic lupus erythematosus (lupus or SLE), myasthenia gravis, multiple sclerosis, psoriasis, spondylitis, Sjogren's syndrome, Graves disease, and Crohn's disease. As used herein, an “inflammatory disease” refers to a disease caused by or resulting from or resulting in inflammation. The term “inflammatory disease” may also refer to a dysregulated inflammatory reaction that causes an exaggerated response by macrophages, granulocytes, mast cells, B-lymphocytes, and/or T-lymphocytes leading to abnormal tissue formation, regeneration, repair (e.g., scaring, fibrosis, keloids), damage and cell death.

The micelles and/or compositions of the present invention can be administered by any suitable route, for example, by injection (e.g., for local (direct) or systemic administration (intravenous)), oral, pulmonary, topical, nasal or other modes of administration. For example, the micelles and/or compositions may be administered by parenteral, intramuscular, intravenous, intraarterial, intraperitoneal, subcutaneous, topical, inhalatory, transdermal, intraocular, intrapulmonary, intrarectal, and intranasal administration. In a particular embodiment, the micelles and/or compositions are injected directly to the desired site or intravenously. In general, the pharmaceutically acceptable carrier of the composition is selected from the group of diluents, preservatives, solubilizers, emulsifiers, adjuvants and/or carriers. The compositions can include diluents of various buffer content (e.g., Tris HCl, acetate, phosphate), pH and ionic strength; and additives such as detergents and solubilizing agents (e.g., Tween® 80, Polysorbate 80), anti oxidants (e.g., ascorbic acid, sodium metabisulfite), preservatives (e.g., Thimersol, benzyl alcohol) and bulking substances (e.g., lactose, mannitol). The pharmaceutical composition of the present invention can be prepared, for example, in liquid form, or can be in dried powder form (e.g., lyophilized).

In yet another embodiment, the compositions of the present invention can be delivered in a controlled release system, such as using an intravenous infusion, an implantable osmotic pump, a transdermal patch, or other modes of administration. In a particular embodiment, a pump may be used. In another embodiment, polymeric materials may be employed (see Medical Applications of Controlled Release, Langer and Wise (eds.), CRC Press: Boca Raton, Fla.; Controlled Drug Bioavailability, Drug Product Design and Performance, Smolen and Ball (eds.), Wiley: New York). In yet another embodiment, a controlled release system can be placed in proximity of the target tissues of the animal, thus requiring only a fraction of the systemic dose. In particular, a controlled release device can be introduced into an animal in proximity to the site of bone fracture.

The dosage ranges for the administration of the composition of the invention are those large enough to produce the desired effect (e.g., curing, relieving, and/or preventing the bone disease, disorder, or fracture, the symptom of it, or the predisposition towards it). The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counter indications. An effective amount of a drug is well known in the art and changes due to the age, weight, severity of a subject's condition, the particular compound in use, the strength of the preparation, and the mode of administration. The determination of an effective amount is preferably left to the prudence of a treating physician, but may be determined using methods well known in the art (The Pharmacological Basis of Therapeutics, Gilman et al. eds., McGraw-Hill Press; Remington's Pharmaceutical Science's, Easton: Mack Publishing Co.). The compositions of the invention may be prepared using methods known in the art, for example, the preparation of a pharmaceutical composition is known in the art (The Pharmacological Basis of Therapeutics, Gilman et al. eds., McGraw-Hill Press; Remington's Pharmaceutical Science's, Easton: Mack Publishing Co.).

### DEFINITIONS

The singular forms “a,” “an,” and “the” include plural referents unless the context clearly dictates otherwise.

As used herein, the term “agonist” refers to an agent (e.g., protein, polypeptide, peptide, lipid, antibody, antibody fragment, large molecule, or small molecule) that binds to a receptor and has an intrinsic effect such as inducing a receptor-mediated response. For example, the agonist may stimulate, increase, activate, facilitate, enhance, or up regulate the activity of the receptor.

The term “treat” as used herein refers to any type of treatment that imparts a benefit to a patient afflicted with a disease or disorder, including improvement in the condition of the patient (e.g., in one or more symptoms), delay in the progression of the condition, etc.

The phrase “effective amount” refers to that amount of therapeutic agent that results in an improvement in the patient's condition.

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, adjuvant, preservative (e.g., Thimersol, benzyl alcohol), anti-oxidant (e.g., ascorbic acid, sodium metabisulfite), solubilizer (e.g., Tween® 80, Polysorbate 80), emulsifier, buffer (e.g., Tris HCl, acetate, phosphate), bulking substance (e.g., lactose, mannitol), excipient, auxilliary agent or vehicle with which an active agent of the present invention is administered. Pharmaceutically acceptable carriers can be sterile liquids, such as water and oils, including those of petroleum, animal, vegetable or synthetic origin, such as peanut oil, soybean oil, mineral oil, sesame oil and the like. Water or aqueous saline solutions and aqueous dextrose and glycerol solutions are preferably employed as carriers, particularly for injectable solutions. The compositions can be incorporated into particulate preparations of polymeric compounds such as polylactic acid, polyglycolic acid, etc., or into liposomes or micelles. Such compositions may influence the physical state, stability, rate of in vivo release, and rate of in vivo clearance of components of a pharmaceutical composition of the present invention. The pharmaceutical composition of the present invention can be prepared, for example, in liquid form, or can be in dried powder form (e.g., lyophilized). Suitable pharmaceutical carriers are described in “Remington's Pharmaceutical Sciences” by E.W. Martin (Mack Publishing Co., Easton, Pa.); Gennaro, A. R., Remington: The Science and Practice of Pharmacy, (Lippincott, Williams and Wilkins); Liberman, et al., Eds., Pharmaceutical Dosage Forms, Marcel Decker, New York, N.Y.; and Kibbe, et al., Eds., Handbook of Pharmaceutical Excipients, American Pharmaceutical Association, Washington.

The term “isolated” refers to the separation of a compound from other components present during its production. “Isolated” is not meant to exclude artificial or synthetic mixtures with other compounds or materials, or the presence of impurities that do not substantially interfere with the fundamental activity, and that may be present, for example, due to incomplete purification, or the addition of stabilizers.

The terms “linker”, “linker domain”, and “linkage” refer to a chemical moiety comprising a covalent bond or a chain of atoms that covalently attaches at least two compounds, for example, a water soluble polymer to a therapeutic agent. The linker can be linked to any synthetically feasible position of the compounds, but preferably in such a manner as to avoid blocking the compounds desired activity. The linker may be biodegradable or non-degradable under physiological environments or conditions.

As used herein, the term “biodegradable” or “biodegradation” is defined as the conversion of materials into less complex intermediates or end products by solubilization hydrolysis under physiological conditions, or by the action of biologically formed entities which can be enzymes or other products of the organism. The term “non-degradable” refers to a chemical structure that cannot be cleaved under physiological condition, even with any external intervention. The term “degradable” refers to the ability of a chemical structure to be cleaved via physical (such as ultrasonication), chemical (such as pH of less than 6 or more than 8) or biological (enzymatic) means.

As used herein, the term “bone-targeting” refers to the capability of preferentially accumulating in hard tissue rather than any other organ or tissue, after administration in vivo.

As used herein, the term “subject” refers to an animal, particularly a mammal, particularly a human.

As used herein, the term “bone fracture” refers to bone that has been cracked or broken.

The term “alkyl,” as employed herein, includes straight, branched, and cyclic chain hydrocarbons containing 1 to about 20 carbons or 1 to about 10 carbons in the normal chain. The hydrocarbon chain of the alkyl groups may be interrupted with one or more oxygen, nitrogen, or sulfur. Examples of alkyl groups include, without limitation, methyl, ethyl, propyl, isopropyl, butyl, t-butyl, isobutyl, pentyl, hexyl, isohexyl, heptyl, 4,4 dimethylpentyl, octyl, 2,2,4 trimethylpentyl, nonyl, decyl, the various branched chain isomers thereof, and the like. Each alkyl group may, optionally, be substituted (e.g., with 1 to about 5 substituents). The term “lower alkyl” refers to an alkyl which contains 1 to 4 carbons in the hydrocarbon chain. The term “cyclic alkyl” or “cycloalkyl,” as employed herein, includes cyclic hydrocarbon groups containing 1 to 3 rings which may be fused or unfused. Cycloalkyl groups may contain a total of 3 to 20 carbons forming the ring(s), particularly 6 to 10 carbons forming the ring(s). Optionally, one of the rings may be an aromatic ring as described below for aryl. The cycloalkyl groups may also, optionally, contain substituted rings that includes at least one (e.g., from 1 to about 4) sulfur, oxygen, or nitrogen heteroatom ring members. Each cycloalkyl group may be, optionally, substituted, with 1 to about 5 substituents. Alkyl substituents include, without limitation, alkyl, alkenyl, halo (such as F, Cl, Br, I), haloalkyl (e.g., CCl₃ or CF₃), alkoxyl, alkylthio, hydroxy, methoxy, carboxyl, oxo, epoxy, alkyloxycarbonyl, alkylcarbonyloxy, amino, carbamoyl (e.g., NH₂C(═O)— or NHRC(═O)—, wherein R is an alkyl), urea (—NHCONH₂), alkylurea, aryl, ether, ester, thioester, nitrile, nitro, amide, carbonyl, carboxylate and thiol. The alkyl group may be unsaturated. “Alkenyl” refers to an unsubstituted or substituted hydrocarbon moiety (e.g., alkyl) comprising one or more carbon to carbon double bonds (i.e., the alkenyl group is unsaturated) and containing from 1 to about 20 carbon atoms or from 1 to about 10 carbon atoms, which may be a straight, branched, or cyclic hydrocarbon group. The hydrocarbon chain of the alkenyl groups may be interrupted with one or more oxygen, nitrogen, or sulfur. When substituted, alkenyl groups may be substituted at any available point of attachment.

The term “aryl,” as employed herein, refers to monocyclic and bicyclic aromatic groups containing 6 to 10 carbons in the ring portion. Examples of aryl groups include, without limitation, phenyl, naphthyl, such as 1-naphthyl and 2-naphthyl, indolyl, and pyridyl, such as 3-pyridyl and 4-pyridyl. Aryl groups may be optionally substituted through available carbon atoms, preferably with 1 to about 4 groups. Exemplary substituents are described above for alkyl groups. The aryl groups may be interrupted with one or more oxygen, nitrogen, or sulfur heteroatom ring members (e.g., a heteroaryl).

The following example is provided to illustrate certain embodiments of the invention. It is not intended to limit the invention in any way.

### EXAMPLE

### Materials and Methods

### Materials

Simvastatin was purchased from Zhejiang Ruibang Laboratories (Wenzhou, Zhejiang, China). 3-Butyn-1-ol was purchased from Matrix Scientific (Columbia, S.C.). Polyethylene glycol monomethylether (mPEG, 1.9 kDa) was purchased from Alfa Aesar (Ward Hill, Mass.). Heterofunctional PEG (NH₂-PEG-N₃, 3 kDa) was purchased from Rapp Polymere (Tubingen, Germany). IRDye® 800CW NHS ester infrared dye was purchased from LI-COR Biosciences (Lincoln, Nebr.). All other reagents and solvents were purchased from Acros Organics (Morris Plains, N.J.) and used as received.

### Synthesis of the Amphiphilic Macromolecular Prodrug SIM-mPEG

As shown in FIG. 1, to synthesize the micelle-forming macromolecular prodrug of SIM, the hydroxyl group of mPEG (1.9 kDa) was converted to azide via an iodination step, providing one of the coupling synthons for the final “click” reaction. Catalyzed by tosylic acid (TsOH), SIM reacted with 3-butyn-1-ol to give the ring-opened compound 4. The hydroxyl groups of compound 4 further reacted with succinic anhydride to produce the dicarboxylic acid compound 5. It was coupled with two additional SIMs to give compound 6, which was composed of three SIM units and an alkyne handle. The final compound 7 (SIM-mPEG) was successfully obtained by a “click” reaction between azide compound 3 and alkyne compound 6.

### Synthesis of α-Methoxy-Co-Iodo-PEG (Compound 2)

mPEG (1.9 kDa, 3.8 g, 2 mmol) was dissolved in anhydrous dichloromethane (DCM, 40 mL) and cooled in an ice-water bath. Under the protection of argon (Ar), triphenyl phosphine (2.62 g, 10 mmol) was added at 0° C., and the solution was stirred for 10 minutes. Iodine (2 g, 8 mmol) was added in several portions at 0° C., then the temperature of the solution was raised to room temperature and stirred for 24 hours. A total of 20 mL of saturated Na₂SO₃ solution was added and stirred for 10 minutes. The organic phase was then separated and dried over anhydrous Na₂SO₄. The product was then loaded on a silica gel column and eluted with DCM and ethyl acetate (EtOAc) (1:1) and then pure EtOAc to remove the byproduct. Lastly, DCM and methanol (1:1) were used as eluent to obtain the product 3.16 g, yield: 79.1%. For NMR analyses of all the new compounds synthesized, a Varian Inova Unity 500 NMR Spectrometer was used. ¹H-NMR (500 MHz, CDCl₃): δ (ppm)=3.75 (t, J=6.8 Hz, 2H), 3.65 (br, 164H), 3.38 (s, 3H), 3.26 (t, J=6.9 Hz, 2H). ¹³C-NMR (125 MHz, CDCl₃): δ (ppm)=71.33, 69.97 (br), 69.62, 58.42, 2.57.

### Synthesis of α-Methoxy-Co-Azido-PEG (Compound 3)

Compound 2 (500 mg, 0.25 mmol) and sodium azide (325 mg, 5 mmol) were dissolved in anhydrous dimethylformamide (DMF, 4 mL). The solution was stirred at 100° C. for 24 hours under the protection of Ar. DCM (100 mL) was added and washed with brine. The organic phase was dried and concentrated. The residue was loaded on a short silica gel column and eluted with DCM:MeOH=1:1 to remove the salt. The solvent was evaporated and the residue was further purified by LH-20 column to give the final product 450 mg. Yield: 93.4%. ¹H-NMR (500 MHz, CDCl₃): δ (ppm)=3.77 (t, J=5.0 Hz, 2H), 3.65 (br, 159H), 3.55 (t, J=5.0 Hz, 2H), 3.50 (t, J=5.0 Hz, 2H), 3.46 (t, J=5.0 Hz, 2H), 3.45 (s, 3H). ¹³C-NMR (125 MHz, CDCl₃): δ (ppm)=71.68, 70.44 (br), 70.42, 70.32, 70.26, 69.78, 58.76, 50.42.

### Synthesis of (3R,5R)-but-3-yn-1-yl 7-((1S,2S,6R,8S,8aR)-8-(2,2-dimethylbutanoyl)oxy)-2,6-dimethyl-1,2,6,7,8,8a-hexahydronaphthalen-1-yl)-3,5-dihydroxyheptanoate (compound 4)

Simvastatin (418 mg, 1 mmol) and TsOH monohydrate (19 mg, 0.1 mmol) were dissolved in 3-butyn-1-ol (420 mg, 6 mmol) and stirred at room temperature for 3 hours. Ethyl acetate (50 mL) was added and then washed with saturated NaHCO₃ (5 mL) and brine (20 mL). The aqueous phase was extracted 3 times with ethyl acetate (20 mL). The combined organic phase was dried by anhydrous sodium sulfate and then the solvent was evaporated. Toluene (30 mL) was added to the residue and then evaporated to remove the 3-butyn-1-ol. The residue was purified by flash chromatography (EtOAc:hexanes=1:1 to 3:1), 148 mg of final product was obtained and 252 mg of unreacted simvastatin was recovered. Yield: 30.3%. ¹H-NMR (500 MHz, CDCl₃): δ (ppm)=5.98 (d, J=9.75 Hz, 1H), 5.78 (dd, J=9.75 Hz, 6.34 Hz, 1H), 5.49 (br, 1H), 5.39 (d, J=2.92 Hz, 1H), 4.27 (m, 1H), 4.23 (t, J=6.83 Hz, 2H), 3.98 (s, 1H), 3.78 (m, 1H), 3.68 (s, 1H), 2.55 (td, J=6.83 Hz, 2.44 Hz, 2H), 2.53 (d, J=2.93 Hz, 1H), 2.51 (s, 1H), 2.44 (m, 1H), 2.37 (dd, J=11.71 Hz, 6.34 Hz, 1H), 2.24 (dd, J=11.71 Hz, 2.44 Hz, 1H), 2.02 (t, J=2.44 Hz, 1H), 1.94 (m, 1H), 1.94 (s, 1H), 1.50-1.64 (m, 8H), 1.21 (m, 1H), 1.12 (s, 3H), 1.11 (s, 3H), 1.09 (d, J=7.31 Hz, 3H), 0.87 (d, J=7.32 Hz, 3H), 0.82 (t, J=7.32 Hz, 3H). ¹³C-NMR (125 MHz, CDCl₃): δ (ppm)=178.01, 171.80, 132.99, 131.50, 129.44, 128.20, 79.82, 72.08, 70.02, 68.87, 67.99, 62.17, 42.85, 42.24, 41.74, 37.62, 36.08, 34.70, 32.94, 32.88, 30.38, 27.18, 24.69, 24.58, 24.10, 22.99, 18.81, 13.79, 9.20. MS (ESI): m/z=511 (M+Na⁺), calculated MW=488.

### Synthesis of 4,4′-(((3R,5R)-1-(but-3-yn-1-yloxy)-7-((1S,2S,6R,8S,8aR)-8-(2,2-dimethylbutanoyl)oxy)-2,6-dimethyl-1,2,6,7,8,8a-hexahydronaphthalen-1-yl)-1-oxoheptane-3,5-diyl)bis(oxy))bis(4-oxobutanoic acid) (compound 5)

The diol compound 4 (300 mg, 0.6 mmol) and succinic anhydride (360 mg, 3.6 mmol) were dissolved in anhydrous DMF (10 mL). Triethylamine (TEA, 240 mg, 2.4 mmol) and 4-dimethylaminopyridine (DMAP, 29.28 mg, 0.24 mmol) were added. The solution was stirred at 45° C. for 20 hours. Dilute hydrochloride (0.1 M, 30 mL) was added, followed by 100 mL of EtOAc. The solution was washed with brine and then dried. Flash chromatography separation gave 402 mg of the final product. Yield: 93.4%. ¹H-NMR (500 MHz, CDCl₃): δ (ppm)=10.53 (br, 2H), 5.97 (d, J=8.9 Hz, 1H), 5.76 (dd, J=9.27 Hz, 6.34 Hz, 1H), 5.48 (s, 1H), 5.36 (s, 1H), 5.25 (m, 1H), 4.90 (m, 1H), 4.18 (t, J=6.83 Hz, 2H), 2.67 (t, J=6.34 Hz, 4H), 2.64 (t, J=6.34, 2H), 2.63 (d, J=6.34, 2H), 2.59 (t, J=6.34, 2H), 2.51 (td, J=6.83 Hz, 2.44 Hz, 2H), 2.42 (m, 1H), 2.35 (q, J=5.86, 1H), 2.22 (d, J=12.19 Hz, 2H), 2.02 (t, J=2.44 Hz, 1H), 1.94 (br, 2H), 1.89 (m, 2H), 1.60-1.70 (m, 2H), 1.40-1.60 (m, 3H), 1.33 (m, 1H), 1.11 (s, 6H), 1.07 (d, J=7.31 Hz, 3H), 0.86 (d, J=6.83 Hz, 3H), 0.82 (t, J=7.31 Hz, 3H). ¹³C-NMR (125 MHz, CDCl₃): δ (ppm)=177.98, 177.53, 176.95, 171.65, 171.16, 169.68, 132.72, 131.36, 129.52, 128.20, 79.86, 71.50, 69.94, 67.99, 67.95, 62.29, 42.84, 38.22, 37.58, 37.37, 36.10, 32.79, 32.76, 31.06, 30.30, 28.92, 28.74, 28.70, 28.29, 27.10, 24.54, 24.51, 23.53, 22.90, 18.69, 13.62, 9.13. MS (ESI): m/z=711 (M+Na⁺), calculated MW=688.

### Synthesis of Simvastatin Trimer (Compound 6)

Compound 5 (495 mg, 0.719 mmol) and N,N′-dicyclohexylcarbodiimide (DCC, 370.5 mg, 1.799 mmol) were dissolved in DCM (5 mL) at 0° C. The solution was stirred for 5 min, then DMAP (5.2 mg, 0.043 mmol) and simvastatin (750 mg, 1.799 mmol) were added. The solution was stirred at 0° C. for about 1.5 hours, then diluted with EtOAc and washed with brine. After drying with Na₂SO₄, the solution was evaporated and the residue was purified by flash chromatography (ethyl acetate/hexanes=1:3) to give 0.627 g of the final product with 287 mg of unreacted simvastatin recovered. Yield 58.6%. ¹H-NMR (500 MHz, CDCl₃): δ (ppm)=5.98 (d, J=9.75 Hz, 2H), 5.97 (d, J=9.27 Hz, 1H), 5.76 (m, 3H), 5.51 (m, 3H), 5.36 (d, J=2.44 Hz, 2H), 5.33 (d, J=2.93 Hz, 1H), 5.27 (m, 2H), 5.22 (m, 1H), 4.87 (m, 1H), 4.50 (m, 2H), 4.19 (t, J=6.24 Hz, 2H), 2.79 (dd, J=8.05 Hz, 5.36 Hz, 2H), 2.72 (m, 1H), 2.68 (m, 1H), 2.56-2.66 (m, 10H), 2.53 (td, J=6.83 Hz, 2.44 Hz, 2H), 2.44 (m, 3H), 2.36 (m, 3H), 2.25 (d, J=11.70 Hz, 2H), 2.23 (d, J=12.7 Hz, 1H), 2.09 (m, 2H), 2.02 (t, J=2.44 Hz, 1H), 1.93-1.98 (m, 7H), 1.60-1.90 (m, 10H), 1.40-1.60 (m, 10H), 1.20-1.40 (m, 4H), 1.13 (s, 6H), 1.12 (s, 6H), 1.11 (s, 3H), 1.10 (s, 3H), 1.08 (d, J=7.31 Hz, 6H), 1.07 (d, J=5.37 Hz, 3H), 0.89 (d, J=6.83 Hz, 6H), 0.87 (d, J=6.83 Hz, 3H), 0.827 (t, J=7.32 Hz, 6H), 0.822 (t, J=7.32 Hz, 3H). ¹³C-NMR (125 MHz, CDCl₃): δ (ppm)=177.54, 177.40, 171.59, 171.35, 171.19, 171.01, 169.52, 168.65, 168.61, 132.73, 132.69, 132.68, 131.44, 131.39, 131.38, 129.65, 129.60, 128.31, 79.89, 76.48, 71.49, 69.97, 68.06, 67.83, 67.71, 65.78, 65.73, 62.29, 42.87, 42.81, 38.25, 37.68, 37.39, 36.55, 36.26, 35.22, 33.19, 33.16, 32.95, 32.88, 32.73, 31.11, 30.53, 30.40, 28.87, 27.18, 24.72. MS (ESI): m/z=1511 (M+Na⁺), calculated MW=1488.

### Synthesis of SIM-mPEG (Compound 7)

The simvastatin trimer compound 6 (772 mg, 0.520 mmol), mPEG-N₃ (400 mg, 0.208 mmol), copper sulfate pentahydrate (52 mg, 0.208 mmol) were added to a solution of t-BuOH (3 mL) and water (2 mL). After the solution was bubbled with Ar for 2 minutes, L-ascorbic acid sodium salt (82 mg, 0.416 mmol) was added. The solution was stirred at room temperature for 60 h under the protection of Ar. DCM (100 mL) was added and washed with a solution (186 mg EDTA disodium and 42 mg NaHCO₃ in 30 mL water) and brine (40 mL×3) to remove the copper catalyst. The aqueous phase was extracted with DCM (50 mL). The combined organic phase was concentrated. The residue was loaded on a silica gel column and eluted with EtOAc to recover the unreacted simvastatin trimer (260 mg) and then eluted with a mixed solution (DCM:MeOH=1:1) to yield the final product. After lyophilization, 548 mg white solid was obtained. Yield: 77.3%. ¹H-NMR (500 MHz, CDCl₃): δ (ppm)=7.55 (s, 1H), 5.98 (d, J=9.75, 2H), 5.97 (d, J=9.25, 1H), 5.76 (m, 3H), 5.50 (m, 3H), 5.37 (d, J=2.93 Hz, 2H), 5.33 (m, 1H), 5.27 (m, 2H), 5.22 (m, 1H), 4.87 (m, 1H), 4.50 (t, J=5.37 Hz, 2H), 4.48 (m, 2H), 4.36 (t, J=6.83 Hz, 2H), 3.88 (t, J=5.37 Hz, 2H), 3.77 (t, J=4.87 Hz, 2H), 3.62 (br, 164H), 3.54 (t, J=4.88 Hz, 2H), 3.37 (s, 3H), 3.05 (t, J=7.31 Hz, 2H), 2.78 (dd, J=8.04 Hz, 5.37 Hz, 2H), 2.70 (m, 2H), 2.68 (m, 1H), 2.56-2.66 (m, 12H), 2.43 (m, 3H), 2.36 (m, 3H), 2.25 (dd, J=12.20 Hz, 2H), 2.23 (d, J=12.7 Hz, 1H), 2.08 (m, 2H), 1.93-1.98 (m, 7H), 1.60-1.90 (m, 14H), 1.40-1.60 (m, 9H), 1.20-1.40 (m, 4H), 1.12 (s, 6H), 1.117 (s, 6H), 1.106 (s, 3H), 1.100 (s, 3H), 1.09 (d, J=7.31 Hz, 6H), 1.07 (d, J=5.37 Hz, 3H), 0.89 (d, J=6.83 Hz, 6H), 0.87 (d, J=6.83 Hz, 3H), 0.830 (t, J=7.32 Hz, 6H), 0.821 (t, J=7.32 Hz, 3H). ¹³C-NMR (125 MHz, CDCl₃): δ (ppm)=177.56, 177.42, 171.61, 171.38, 171.22, 171.05, 169.68, 168.70, 168.67, 143.45, 132.77, 132.70, 131.46, 131.40, 129.67, 129.66, 128.32, 122.59, 76.50, 71.85, 70.48 (br), 69.44, 68.07, 67.85, 67.74, 65.84, 65.47, 58.94, 50.05, 42.89, 42.83, 38.31, 37.73, 37.40, 36.56, 36.27, 35.23, 33.19, 32.96, 32.89, 32.74, 32.42, 31.07, 30.54, 30.40, 28.89, 27.19, 26.08, 25.31, 24.74, 24.66, 24.12, 23.51, 22.94, 13.83, 13.77, 9.26.

### Synthesis of IRDye® 800CW-Labeled SIM-mPEG (SIM-mPEG-IRDye®)

The heterofunctional PEG (46.8 mg, 0.01562 mmol) and IRDye 800CW NHS ester (1.214 mg, 0.00104 mmol) were dissolved in anhydrous DMF (1 mL) and bubbled with Ar for 1 minute. After addition of N,N-diisopropylethylamine (DIPEA, 5.38 mg, 0.0416 mmol), the solution was stirred at room temperature for 20 hours. Acetic anhydride (15.9 mg, 0.156 mmol) was then added and stirred for 12 hours. The mixture was purified by LH-20 column to give 45.4 mg final product, which was a mixture of PEG labeled by IRDye® 800CW and PEG capped by an acetyl group. Yield: 97%. ¹H-NMR (500 MHz, CDCl₃): δ (ppm)=6.38 (s, 1H), 3.77 (t, J=5.36 Hz, 2H), 3.63 (br, 301H), 3.55 (t, J=4.88 Hz, 2H), 3.38 (t, J=4.88 Hz, 2H), 1.97 (s, 3H). ¹³C-NMR (125 MHz, CDCl₃): δ (ppm)=170.19, 70.61, 70.58, 70.55, 70.47 (br), 70.08, 69.97, 69.80, 50.58, 39.20, 23.14.

Simvastatin trimer compound 6 (133.9 mg, 0.09 mmol), PEG mixture (45 mg, 0.015 mmol) and copper sulfate pentahydrate (7.5 mg, 0.03 mmol) were dissolved in Ar bubbled solution of t-BuOH (1 mL), H₂O (0.5 mL), DMF (1 mL). The solution was bubbled by Ar for another 1 min and then L-ascorbic acid sodium (11.88 mg, 0.06 mmol) was added. The mixture was stirred at room temperature for 48 hours. The product was purified by LH-20 column chromatography. After lyophilization, 35.7 mg product was obtained. ¹H-NMR (500 MHz, CDCl₃): δ (ppm)=7.56 (s, 1H), 6.46 (s, 1H), 5.98 (d, J=9.75, 2H), 5.97 (d, J=9.25, 1H), 5.76 (m, 3H), 5.50 (m, 3H), 5.37 (d, J=2.93 Hz, 2H), 5.33 (m, 1H), 5.27 (m, 2H), 5.22 (m, 1H), 4.87 (m, 1H), 4.50 (t, J=5.37 Hz, 2H), 4.48 (m, 2H), 4.36 (t, J=6.83 Hz, 2H), 3.88 (t, J=5.37 Hz, 2H), 3.77 (t, J=4.87 Hz, 2H), 3.62 (br, 294H), 3.54 (t, J=4.88 Hz, 2H), 3.37 (s, 3H), 3.05 (t, J=7.31 Hz, 2H), 2.78 (dd, J=8.04 Hz, 5.37 Hz, 2H), 2.70 (m, 2H), 2.68 (m, 1H), 2.56-2.66 (m, 12H), 2.43 (m, 3H), 2.36 (m, 3H), 2.25 (dd, J=12.20 Hz, 2H), 2.23 (d, J=12.7 Hz, 1H), 2.08 (m, 2H), 1.98 (s, 3H), 1.93-1.98 (m, 7H), 1.60-1.90 (m, 14H), 1.40-1.60 (m, 9H), 1.20-1.40 (m, 4H), 1.12 (s, 6H), 1.117 (s, 6H), 1.106 (s, 3H), 1.100 (s, 3H), 1.09 (d, J=7.31 Hz, 6H), 1.07 (d, J=5.37 Hz, 3H), 0.89 (d, J=6.83 Hz, 6H), 0.87 (d, J=6.83 Hz, 3H), 0.830 (t, J=7.32 Hz, 6H), 0.821 (t, J=7.32 Hz, 3H). ¹³C-NMR (125 MHz, CDCl₃): δ (ppm)=177.56, 177.44, 171.62, 171.38, 171.22, 171.06, 170.15, 169.69, 168.68, 143.49, 132.71, 131.47, 131.41, 129.68, 128.33, 122.64, 76.51, 71.52, 70.49 (br), 70.12, 69.79, 69.44, 68.08, 67.86, 67.74, 65.80, 65.76, 63.47, 50.09, 42.90, 42.84, 39.23, 38.33, 37.75, 37.41, 36.57, 36.28, 35.25, 33.20, 32.97, 32.91, 32.76, 32.00, 30.55, 30.41, 28.90, 27.21, 25.31, 24.75, 24.67, 24.13, 23.52, 23.14, 22.96, 13.84, 13.78, 9.27.

The IRDye® 800CW content was determined as 1.34±0.053 μmol/g in the final product using a NanoDrop™ 2000 UV-Vis Spectrophotometer (Thermo Scientific, Wilmington, Del., USA).

### Micelle Formulation, Characterization and Free SIM Loading Micelle Formulation

Two different methods were used to prepare the free simvastatin-loaded micelles (SIM/SIM-mPEG). For the film hydration method, SIM (10 mg) was dissolved in a methanol solution of SIM-mPEG (1 mL, 10 mg/mL). Methanol was then removed by rotor evaporation at 60° C. to form a film in the round bottom flask, which was placed in vacuum overnight to remove any residue solvent. To prepare the micelle, the film was hydrated by distilled water at room temperature for 30 minutes. For the direct dissolution method, SIM-mPEG was first dissolved in water (1 mL, 10 mg/mL) and placed at 4° C. overnight, and then the system was equilibrated at 24° C. for 24 hours in order to allow micelle formation. SIM (10 mg) was then added to this solution, and dissolution of the drug was allowed under stirring for 24 hours at room temperature. In both cases, the undissolved SIM was removed by centrifugation at 12,000 rpm for 0.5 minutes, followed by filtration of the supernatant through a 0.2 μm filter. The direct dissolution of SIM-mPEG alone in water was utilized as a control.

### Characterization of the Micelles and Drug Loading Efficiency

Effective hydrodynamic diameters (D_{eff}), polydispersity index (PDI) and ζ-potential of the micelles were measured by dynamic light scattering (DLS) using a Zetasizer Nano ZS90 (Malvern Instruments, Worcestershire, UK). To estimate loading efficiency, the undissolved SIM was collected and quantified by an Agilent 1100 HPLC system with a quaternary pump (with degasser), an autosampler and a diode-array based UV detector. The loading efficiency was calculated by subtracting the amount of undissolved SIM from the total amount of SIM used to prepare the micelle formulation.

### In Vivo Evaluation of the Targeting and Therapeutic Efficacy of SIM/SIM-mPEG Micelles on Fracture Model

### Establishment of the Open Fracture Mouse Model

Ten-week-old male Swiss Webster mice were purchased from Charles River Laboratories, and maintained under standard housing conditions. The animals were acclimated for at least 1 week before any experimental procedure. Under general anesthesia with isoflurane, the hips, thighs, and knees on the right side of the animal were prepared with Betadine (povidone-iodine) solution. A 1.0 cm right medial parapatellar incision was made on the right knee. The patella was dislocated laterally to expose the femoral condyles. A 25-guage needle was introduced into the canal and driven in a retrograde intramedullary fashion to the level of the greater trochanter. Distally, the needle was cut flush with the cortex (length was about 1.4 cm) so as not to interfere with motion of the knee. Controlled unilateral transverse femoral shaft fractures were created with a #15 blade. The extensor mechanism was closed with interrupted absorbable sutures (size 4-0) in a standard fashion followed by closure of the skin with 4-0 sutures. Postoperatively, mice were placed on clean paper towels in their cages (to avoid inhalation/aspiration of the bedding material) and monitored until the animals were fully awake. The mice were monitored for signs of pain with analgesia and antibiotics given according to established protocol. Contralateral sham osteotomy was not performed due to the concerns of animal welfare. All animal procedures were approved by the Institutional Animal Care and Use Committee (IACUC) of University of Nebraska Medical Center.

### Targeting of SIM/SIM-mPEG Micelles to Fracture Site

To validate the potential of the micelles' passive targeting and retention to the fracture site, a near-infrared (NIR) optical imaging study was performed to observe the in vivo biodistribution of the SIM/SIM-mPEG micelles after its systemic administration. TRDye® 800CW labeled and un-labeled prodrug were mixed to prepare the IRDye-labeled SIM/SIM-mPEG micelles (STM-PEG-TRDye®, 1 mg/mouse, n=3) and then given to fractured mice via tail vein injection 7-day post osteotomy. The mice were imaged prior to and then daily after the micelle administration using a LI-COR Pearl™ Impulse Small Animal Imaging System (Lincoln, Nebr.) to evaluate the distribution of the micelles continuously for 7 days. The images were captured using channels 800 nm and white (optical) with the resolution of 170 μm. The NIR signal intensity was measured semi-quantitatively at a consistent region of interest (ROI) at the fracture and the contralateral site for all the mice.

### Therapeutic Efficacy of SIM/SIM-mPEG Micelles in the Mouse Fracture Model

Eighteen 10-week-old male Swiss Webster mice were osteotomized and randomly assigned into three groups (6 mice/group). At 7 days post osteotomy, the mice were treated with saline (vehicle control or CON), simvastatin acid (SIMA, equiv. SIM 6 mg/kg/day, i.p.) or SIM/SIM-mPEG micelles (equiv. SIM 42 mg/kg/wk, weekly i.v.) by tail vein injection during the treatment period of two weeks. The overall SIM doses given to the SIMA and SIM/SIM-mPEG were equivalent. All mice were euthanized 21 days post osteotomy.

The fractured femurs were collected, fixed in 10% formalin and processed for further micro-CT imaging and analysis (Bruker SkyScan™1172, Kontich, Belgium). Each femur was scanned and reconstructed into a 3D-structure with a voxel size of 5.5 μm. The X-ray tube voltage was 70 kV and the current was 141 μA, with a 0.5 mm thick aluminum filter. Exposure time was 530 ms. The X-ray projections were obtained at 0.7° intervals with a scanning angular rotation of 180° and eight frames were averaged for each rotation. 3D reconstructions were performed using NRecon software.

For the quantitative analysis, we specifically focused on the site of fracture callus formation. The position of each bone was corrected with DataViewer and then saved on a volume of interest (VOI) of 400 slides (resized by 2). This was followed by careful placement of a constant square region of interest (ROI) in the callus with 200 slides above and below the fracture line. To standardize analysis within the animals, special care was taken so as to keep the constant ROI in the center for each sample, with the fracture line and metal rod (fixation needle), respectively serving as horizontal and vertical points of reference. A custom analysis process was performed with series of specific threshold settings in CTan, which were kept constant for all samples. The following parameters were measured and calculated: callus volume (Cl.V), callus thickness (Cl.Th), callus separation (Cl.Sp) and the ratio of callus surface to volume (Cl.S/Cl.V). 3D images were exported using DataViewer to produce a visual representation of the results.

After micro-CT analysis, the fractured femurs were decalcified using 14% EDTA at room temperature for 30 days. The decalcification solution was changed daily. The specimens were then paraffin embedded, sectioned (5 μm thickness), stained with Safranin 0 and histologically evaluated.

### Statistical Analysis

Data were analyzed using ANOVA and Student's t test for comparisons. A value of P<0.05 was considered statistically significant.

### Results

### Micelle Formulation, Characterization and Free SIM Loading.

Due to the unique amphiphilic structural, SIM-mPEG can spontaneously self-assemble into micelles and further incorporate more free SIM molecules into the hydrophobic cores of the micelles. As shown in Table 1, compared with the control, the particle size of the SIM-mPEG micelles increased with the free SIM incorporated into their hydrophobic cores. To optimize the preparation of SIM/SIM-mPEG micelles with proper particle size, narrow size distribution and higher drug loading, the film hydration method was selected to prepare SIM/SIM-mPEG micelles for all the following studies. A high loading capacity of close to 100% (W_{d}/Wₚ) was achieved. The micelle diameter was determined to be around 30 nm, with a PDI value of 0.053 and a ζ-potential of −3.3 mV.

TABLE 1
Characterization of SIM/SIM-mPEG micelles.

| | Drug | | | |
|---|---|---|---|---|
| Prep- | Loading | | | |
| aration | Efficiency | D_{eff} | | ζ-Potential |
| Method | (%) | (nm)^{a} | PDI^{a} | (mV) |
| Film | 99.76 ± 0.04 | 30.09 ± 0.22 | 0.053 ± 0.015 | −3.29 ± 0.29 |
| Hydration | | | | |
| Direct | 34.27 ± 0.78 | 252.03 ± 1.50 | 0.247 ± 0.006 | −10.7 ± 0.3 |
| Dissolu- | | | | |
| tion | | | | |
| Control | N/A | 18.46 ± 1.50 | 0.396 ± 0.013 | −5.96 ± 0.35 |

^{a}Effective diameter (D_{eff}) and polydispersity indices (PDI) were determined by DLS at 25° C. (n = 3).

### Targeting of SIM/SIM-mPEG Micelles to the Fracture Site

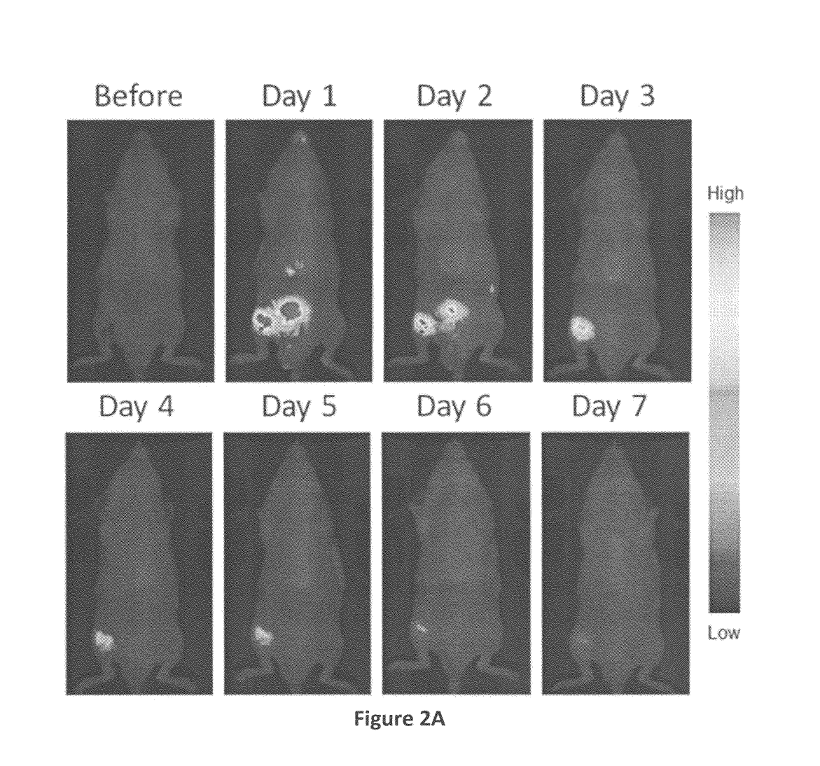

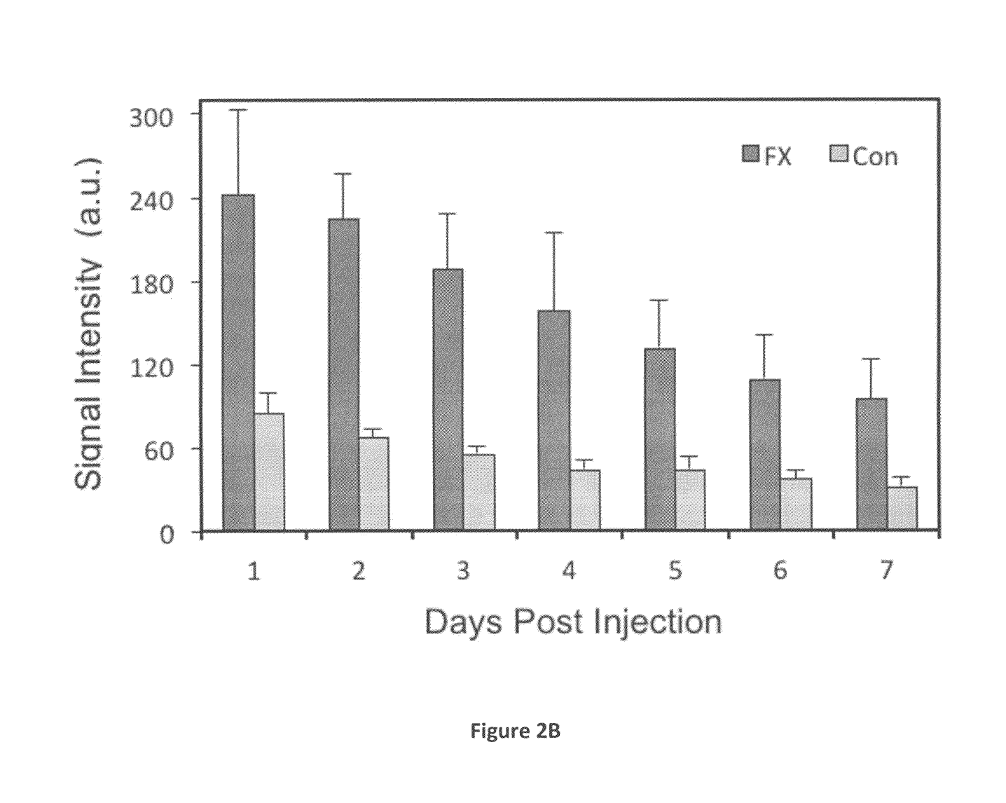

As shown in FIG. 2, compared to the intact contralateral side, the fractured leg demonstrated intense and long-lasting NIR signals in the femoral fracture region, consistent with targeting and sustained retention of the IRDye-labeled SIM/SIM-mPEG micelles at the fracture site. After selection of the region of interest (ROI), the NIR signal intensity was analyzed semi-quantitatively. The signal intensity differences between the fracture site and the intact site were statistically significant (P<0.05) which further confirmed the direct visual observation.

### Therapeutic Efficacy of SIM/SIM-mPEG Micelles on Fracture Healing.

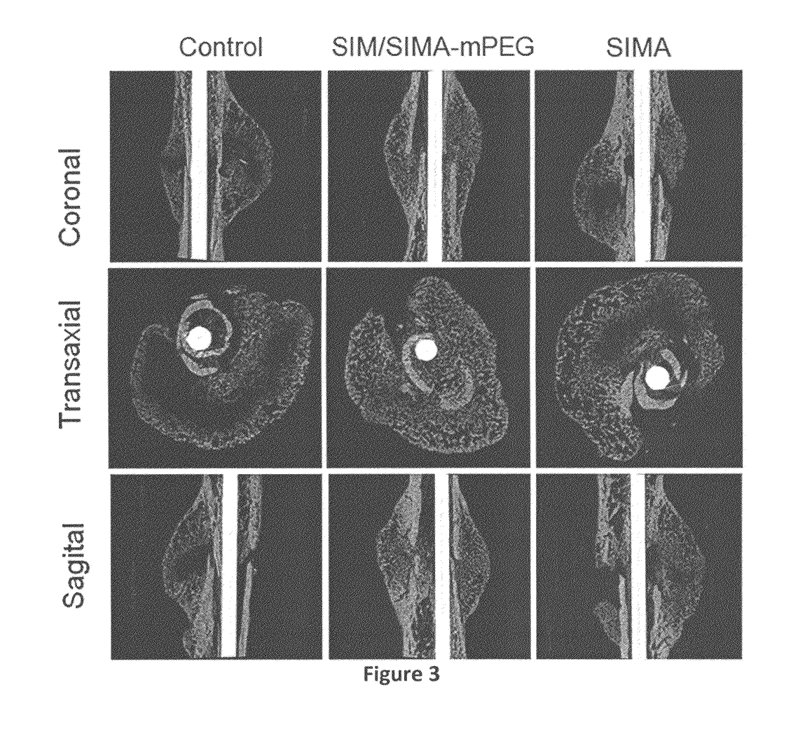

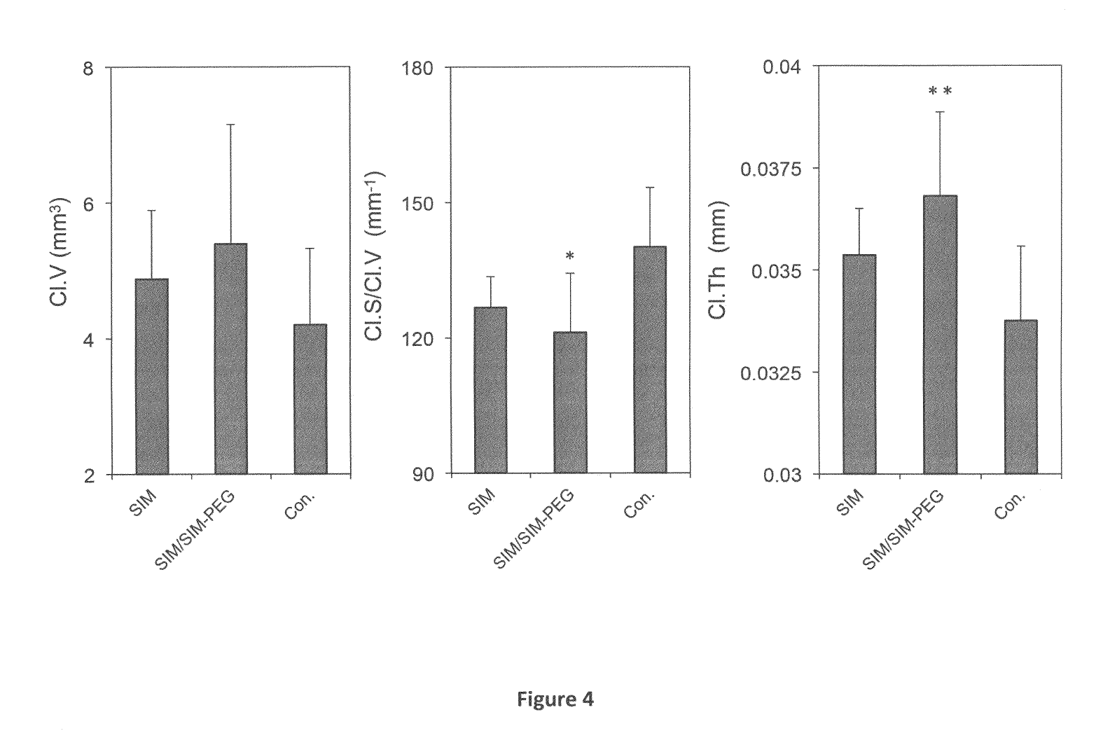

Callus mineralization is an essential feature of fracture repair. As shown in FIG. 3, during the fracture healing process, the SIM/SIM-mPEG treated group exhibited extensive mineralization of the fracture callus. In contrast, the CON and SIMA treated groups demonstrated less mineralized callus formation at the fracture site (shown as dark areas or cavities in FIG. 3). Micro-CT data were further analyzed to quantitatively assess the impact of the different treatments on the histomorphometric parameters of the fracture callus (FIG. 4). Callus volume (Cl.V) and callus thickness (Cl.Th) in the SIM/SIM-mPEG treated mice were higher (+28.6%, P=0.149 and +9.1%, P=0.009, respectively) than the CON mice, while the ratio of callus surface to volume (Cl.S/Cl.V) for SIM/SIM-mPEG group was significantly lower than those of the CON group (−13.5%, P=0.014). The demonstration of increased callus volume, thickness and better organization provides further evidence of enhanced fracture repair in the SIM/SIM-mPEG group. No statistical significance was found in quantitative comparison between the SIMA and CON groups.

### Histological Evidence of Enhanced Fracture Healing in the SIM/SIM-mPEG Micelle Treated Group.

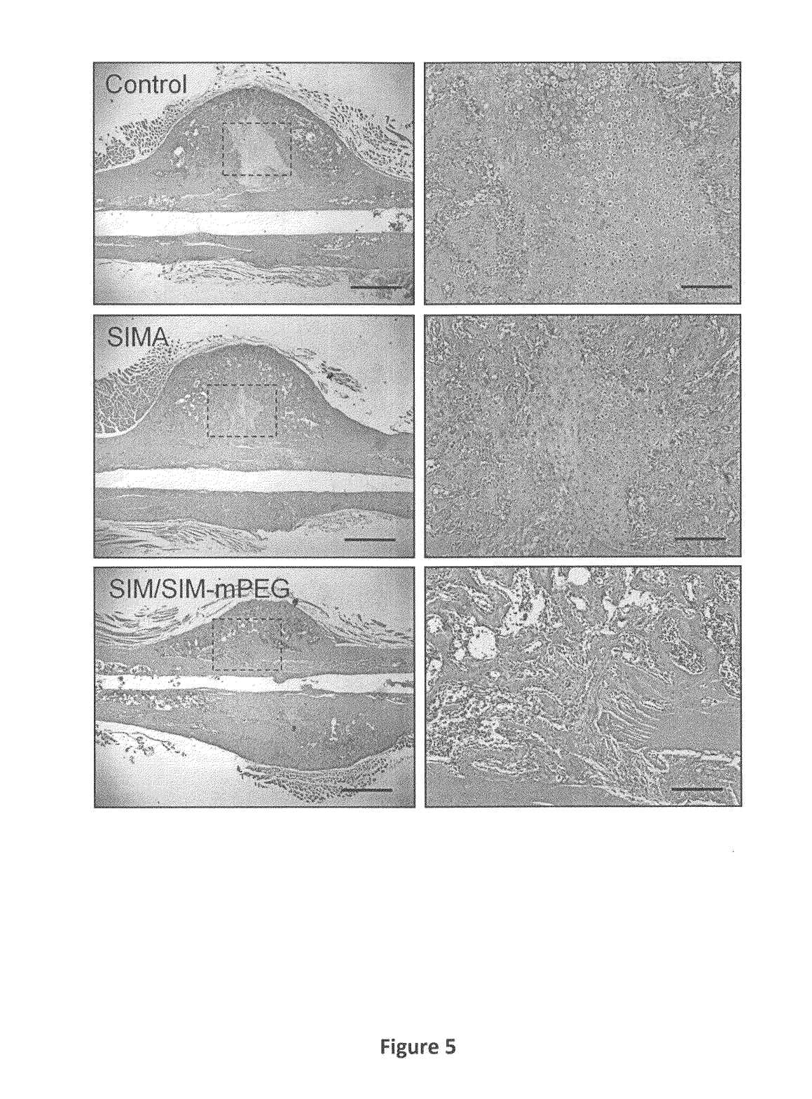

As shown in FIG. 5, at 21 days post fracture, the callus sections from the CON group were populated primarily by cells with morphologic features of chondrocytes surrounded by a proteoglycan rich ECM (stained red) consistent with a relatively early phase of fracture repair. In the callus sections from the animals treated with SIM/SIM-mPEG micelles, however, the fracture site has been replaced with mineralized tissues organized into woven bone, consistent with a more advanced stage of fracture repair. The histological features of SIMA group are consistent with an intermediate stage between CON and SIM/SIM-mPEG groups (Rosen, C. J., American Society for Bone and Mineral Research. Primer on the metabolic bone diseases and disorders of mineral metabolism. 7th ed. Washington, D.C.: American Society for Bone and Mineral Research; 2009, p. 61-2).

Osteoporosis and associated skeletal complications, such as impaired fracture healing and nonunion have placed a significant burden to the public healthcare system in caring for the ever-growing aging population. While several bone anabolic agents have been developed, US FDA has not approved a systemic therapy for the treatment of impaired fracture healing and nonunion. As an anti-lipidaemic drug class, statins have been shown to exhibit a bone anabolic effect (Mundy, G. (1999) Science 286:1946-9; Garrett et al. (2002) Arthritis Res., 4:237-40); however their poor water solubility and strong hepatotropicity (Jadhav et al. (2006) J. Pharm. Pharmacol., 58:3-18; Park, J. B. (2009) Med. Oral Patol. Oral Cir. Bucal., 14:e485-8) have prevented their clinical use to modulate and accelerate the fracture-healing process.

The biologic events associated with bone fracture healing have been well established, which include the sequential development of a hematoma followed by local inflammation, granulation tissue formation, soft callus formation and eventual hard callus formation and remodeling (Marsh et al. (1999) Br. Med. Bull., 55:856-69). The early physiological associated with a fracture, especially the initial hematoma formation and subsequent inflammation, represents a window of opportunity for the delivery of bone anabolic agents to the fracture site for therapeutic intervention. The inflammation associated ELVIS mechanism (Wang et al. (2011) Mol. Pharmaceut., 8:991-3; Ren et al. (2011) Mol. Pharmaceut., 8:1043-51; Yuan et al. (2012) Arthritis Rheumat., 64:4029-39; Yuan et al. (2012) Adv. Drug Del. Rev., 64:1205-19) permits passive targeting and retention of a systemically administered colloidal drug delivery system to the fracture sites. The local cell populations and inflammatory cell infiltrates involved in the dynamic remodeling process at the fracture site may sequester, retain and gradually release the therapeutic agent loaded in the delivery vesicles to modulate the healing process.

To capitalize on this unique pathological feature for the fracture site-specific delivery of statins, a shamrock-like amphiphilic SIM prodrug by conjugating a cluster of three SIMs covalently to the chain terminus of PEG is provided. In designing the hydrophobic SIM trimer block, the ester bond was selected as the chemical linkage as it can not only hold the three SIMs together, but also can be hydrolyzed in vivo by esterase to release the conjugated SIMs. Click chemistry, one of the most efficient coupling methods (Hein et al. (2008) Pharm. Res., 25:2216-30), was used to conjugate the SIM trimer to the mPEG terminus to form the amphiphilic macromolecule. Because of the structural similarity between SIM and the SIM trimer, large amounts of free SIM can be incorporated into the hydrophobic core to form SIM/SIM-mPEG micelles. This novel design not only addresses the poor water solubility of SIM, but also permits high drug-loading capacity. More importantly, as the SIM trimer is situated at the hydrophobic core of the micelle structure, the steric hindrance interferes with the hydrolysis of the ester bond, permitting slow and sustained active drug release, which can then exert its bone anabolic effect at the fracture site.

Using the pyrene-based fluorescence method, the critical micelle concentration (CMC) of SIM-mPEG was determined as ˜10⁻⁶ M, indicating that the micelles are very stable and may retain their size/aggregation in the circulation after systemic administration (Lukyanov et al. (2002) Pharm. Res., 19:1424-9), which allows the micelles' passive targeting to the fracture site as evident by the imaging data. Similar to SIM, SIM-mPEG prodrug was found to stimulate the differentiation (Alkaline Phosphatase or ALP activity) and proliferation (MTT assay) of mouse preosteoblast (pOBs, MC3T3-E1 Subclone 4) into bone forming osteoblasts. More specifically, to evaluate osteogenic effects, mouse pOBs were divided into the vehicle treated-group as negative control (Con.), the dose-dependent simvastatin groups at 10⁻⁷ M and 10⁻⁸ M as positive control (Sim), and the dose-dependent SIM-PEG groups at 10⁻⁶, 10⁻⁷, 10⁻⁸, 10⁻⁹ and 10_{—10} M. After exposing to these different drugs at different time points, the dose responses of SIM-PEG, Sim and Con. on the differentiation (Alkaline Phosphatase activity) and proliferation (MTT assay) of pOBs were examined. The results indicated that SIM-PEG at concentrations from 10⁻⁸ to 10⁻¹⁰ M boosted both differentiation and proliferation of pOBs when compared with the control (P<0.05 or P<0.01) after 6 days. These results provide evidence of SIM-mPEG's activation and release of free SIM which exerts its bone anabolic function.

The data from micro-CT analysis indicates that the SIM/SIM-mPEG micelle treatment leads to increased callus formation and better bone organization/mineralization, compared to an equivalent dose of SIMA. In the histological analysis, the SIM/SIM-mPEG treated group exhibited more extensive mineralization and consolidation of the fracture callus during the fracture healing process when compared with the CON and SIMA treated groups, validating an accelerated healing process. Different from the local delivery approaches where matrix materials were often needed for statin retention and release, the systemically administered SIM/SIM-mPEG micelles passively target, retain and release at the fracture site(s) according to local pathologies (ELVIS mechanism) to modulate the healing process. Therefore, it is especially beneficial for management of concurrent multiple fractures and subclinical fractures.

In conclusion, an amphiphilic shamrock-like simvastatin-methoxy polyethylene glycol conjugate (SIM-mPEG) was successfully synthesized which spontaneously self-assembles into stable micelles. Upon formulation with free simvastatin (SIM), the SIM/SIM-mPEG micelles demonstrated selective targeting to the fracture site in a femur osteotomy mouse model. As a direct result of the alteration of SIM's pharmacokinetics and biodistribution profile, the targeting and retention of SIM at the fracture site led to accelerated fracture healing, as evident in the micro-CT and histological evaluation. Moreover, the side effect of weight loss observed with simvastatin acid administration was greatly reduced with SIM-PEG. The success of this formulation corroborates the novel shamrock-like macromolecular prodrug design as a viable strategy to solubilize very hydrophobic compounds. The colloidal nature of the formulation would also impart tropism to the payloads, facilitating in vivo passive targeting to sites of tissue pathology.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. An amphiphilic compound comprising at least three hydrophobic bone anabolic agents attached to the termini of a single water-soluble polymer, wherein said hydrophobic bone anabolic agents are attached to said water-soluble polymer via a linker.

2. The amphiphilic compound of claim 1, wherein said hydrophobic bone anabolic agents are statins.

3. The amphiphilic compound of claim 2, wherein said statin is simvastatin or lovastatin.

4. The amphiphilic compound of claim 2, wherein said statin is simvastatin.

5. The amphiphilic compound of claim 1, wherein said water-soluble polymer is polyethylene glycol or a derivative thereof.

6. The amphiphilic compound of claim 5, wherein said polyethylene glycol derivative is polyethylene glycol monomethylether.

7. The amphiphilic compound of claim 1, wherein said linker is biodegradable.

8. The amphiphilic compound of claim 7, wherein said linker comprises an ester bond.

9. The amphiphilic compound of claim 1, which is compound 7 of FIG. 1.

10. A micelle comprising at least one amphiphilic compound of claim 1.

11. The micelle of claim 1, further comprising a free hydrophobic therapeutic or imaging agent in the core of the micelle.

12. The micelle of claim 11, wherein said free hydrophobic therapeutic agent is a free hydrophobic bone anabolic agent.

13. The micelle of claim 12, wherein said free hydrophobic bone anabolic agent is the same as the hydrophobic bone anabolic agent attached to said water-soluble polymer.

14. The micelle of claim 12, wherein said free hydrophobic bone anabolic agent is different than the hydrophobic bone anabolic agent attached to said water-soluble polymer.

15. A composition comprising the micelle of claim 10 and a pharmaceutically acceptable carrier.

16. A method of increasing bone mass in a subject, said method comprising administering a therapeutically effective amount of at least one micelle of claim 10 to said subject.

17. The method of claim 16, wherein said micelle is administered intravenously.

18. A method of treating a bone fracture in a subject, said method comprising administering a therapeutically effective amount of at least one micelle of claim 10 to said subject.

19. The method of claim 18, wherein said micelle is administered intravenously.

20. A method of treating an autoimmune disease in a subject, said method comprising administering a therapeutically effective amount of at least one micelle of claim 10 to said subject.

21. The method of claim 20, wherein said micelle is administered intravenously.
